(19) United States
(12) Patent Application Publication
(10) Pub. No.: US 2016/0038283 A1
(43) Pub. Date: Feb. 11, 2016

(54) SYSTEMS AND METHODS UTILIZING EXPANDABLE TRANSCATHETER VALVE

(71) Applicant: THE UNIVERSITY OF IOWA RESEARCH FOUNDATION, Iowa City, IA (US)

(72) Inventors: Abhay DIVEKAR, Coralville, IA (US); Osamah ALDOSS, Coralville, IA (US); Joseph TUREK, Iowa City, IA (US); Peter GRUBER, Iowa City, IA (US)

(73) Assignee: THE UNIVERSITY OF IOWA RESEARCH FOUNDATION, Iowa City, IA (US)

(21) Appl. No.: 14/819,489

(22) Filed: Aug. 6, 2015

Related U.S. Application Data
(60) Provisional application No. 62/033,718, filed on Aug. 6, 2014.

(51) Int. Cl. A61F 2/24

(52) U.S. Cl. CPC A61F 2/2418; A61F 2/2469; A61F 2/243; A61F 2230/0069

(57) ABSTRACT

Apparatus and methods for transcatheter valves. Specific embodiments relate to transcatheter flutter valves configured for pediatric use.

### CROSS-REFERENCE TO RELATED APPLICATION

This application claims priority to U.S. Provisional Patent Application Ser. No. 62/033,718, filed Aug. 6, 2014, the contents of which are herein incorporated by reference.

### BACKGROUND OF THE INVENTION

I. Field of the Invention

Embodiments of the present invention relate to apparatus and methods for transcatheter valves. Specific embodiments relate to transcatheter flutter valves configured for pediatric use.

II. Background of the Invention

### SUMMARY OF THE INVENTION

Congenital heart disease is the most common birth defect and disorders of the pulmonary valve and right ventricular outflow track are the most frequent abnormalities that are noted either in isolation or with multiple associated defects. This is typified by tetralogy of Fallot and its variants. In addition the pulmonary valve is occasionally used to replace the aortic valve in certain forms of congenital heart disease namely congenital aortic stenosis and/or insufficiency (Ross procedure).

These pulmonary valve abnormalities are addressed either by trans-catheter intervention and/or surgery. Frequently, especially during surgery, there is a need to enlarge the orifice of the pulmonary valve (frame or annulus). The consequence is that the patient is left with variable degrees of leakage (regurgitation) with or without obstruction(stenosis). It was believed that the right ventricle can “tolerate” the long-term (chronic) volume (from regurgitation) with or without pressure (from stenosis) overload. Indeed most patients do quite well for several years. There is increasing evidence that chronic volume and/or pressure overload causes slow and progressive right ventricular dysfunction which at some point is irreversible. Although ideally all patients should have a competent and unobstructed pulmonary valve, some patients (pulmonary atresia -pulmonary valve not formed) required pulmonary valve replacement as a neonate. Since the valve does not grow and undergoes deterioration, these patients are also left with chronic volume and/or pressure overload.

Limitations in available sizes, lack of growth, rapid deterioration in children and the need for open heart surgery to place or replace the valve are the major hurdles facing this growing population of patients. Therefore, at the present time, pulmonary valve replacement is postponed for as long as possible. Although recent advances have allowed for transcatheter pulmonary valve implantation without the need for surgery, a large number of patients are excluded because of limitations of existing technology, mainly limited to the size of the patient and need for existing surgical scaffolding.

The presence of a non-regurgitant and non-stenotic pulmonary valve will be well-suited for management of patients with congenital heart disease. With appropriate technology the ability to insert a transcatheter pulmonary valve without the need for surgical intervention may allow for earlier intervention, prevention of progressive right ventricular dysfunction and hopefully improve long-term outcome. Because of the relative noninvasive nature of transcatheter intervention (compared to surgery), the procedure can be repeated to maintain pulmonary valve function with a repeat procedure and minimize the number of surgical procedures in the patient's lifetime.

The existing transcatheter pulmonary valves do not allow for a large number of patients throughout early childhood to benefit from competency of the pulmonary valve. There is a need for developing therapies that would allow for establishing a competent pulmonary valve delivered by transcatheter technology.

As described further below, embodiments of the present invention can provide effective treatment of pulmonary stenosis, including for example, in children.

Any embodiment discussed with respect to one aspect of the invention applies to other aspects of the invention as well.

The embodiments in the one section of this disclosure are understood to be embodiments of the invention that are applicable to all aspects of the invention, including those in other sections of the disclosure.

Certain embodiments include an expandable transcatheter valve comprising an expandable framework, and a tubular member coupled to the expandable framework where the tubular member comprises a first end and a second end; the tubular member is coupled to the expandable framework proximal to the first end; the tubular member is coupled to the expandable framework proximal to the second end at a first coupling location and at a second coupling location; and the first coupling location is approximately 180 degrees radially from the second coupling location when the tubular member is viewed looking toward the second end.

In particular embodiments, the tubular member is coupled to the expandable framework proximal to the second end at only the first coupling location and the second coupling location. In some embodiments, the tubular member comprises a first portion and a second portion; the first portion extends axially along the tubular member and extends clockwise radially from the first coupling location to the second coupling location when the tubular member is viewed looking toward the second end; and the second portion extends axially along the tubular member and extends clockwise radially from the second coupling location to the first coupling location when the tubular member is viewed looking toward the second end.

In specific embodiments, the first portion and the second portion of the tubular member are configured to allow fluid flow from the first end of the tubular member to the second end of the tubular member and restrict fluid flow from the second end of the tubular member to the first end of the tubular member. In certain embodiments, the first portion of the tubular member and the second portion of the tubular member form a flutter valve. In particular embodiments, the expandable framework is configured as a wire stent. In some embodiments, the expandable framework is generally cylindrical. In specific embodiments, the expandable framework is approximately 16-36 mm long.

In certain embodiments, the expandable framework is configured to expand from a smaller first diameter to a larger second diameter; the expandable framework is biased towards the larger second diameter; and the tubular member is initially coupled to the expandable framework when the expandable framework is expanded to the larger second diameter. In particular embodiments, the second diameter is approximately 8-30 mm, including for example, 8 mm, 10 mm, 12 mm, 20 mm or 25 mm. In some embodiments, the tubular member is disposed within the expandable framework. Specific embodiments further comprise an expandable catheter disposed within the expandable framework.

Certain embodiments include a method of inserting a flutter valve into a dysfunctional heart valve, the method comprising: inserting an assembly comprising a catheter and a flutter valve into a dysfunctional heart valve; and retracting the catheter from the dysfunctional heart valve, wherein the flutter valve is retained in the dysfunctional heart valve. In particular embodiments, the catheter has an outer diameter less than or equal to approximately 8 mm, or more particularly 7.3 mm, 6.7 mm, or 6.3 mm, or 6.0 mm. In some embodiments, the flutter valve comprises: an expandable framework; and a tubular member coupled to the expandable framework, where the tubular member comprises a first end and a second end; the tubular member is coupled to the stent proximal to the first end; the tubular member is coupled to the expandable framework proximal to the second end at a first coupling location and at a second coupling location; and the first coupling location is approximately 180 degrees radially from the second coupling location when the expandable framework is viewed looking toward the second end.

In specific embodiments, the dysfunctional heart valve is a pulmonary, tricuspid, or mitral valve. In certain embodiments, the dysfunctional heart valve is in a patient with a weight between 10 kg and 40 kg, or more particularly a weight below 30 kg. In particular embodiments, the dysfunctional heart valve is in a patient younger than 10 years of age, or more particularly younger than 5 years of age. Some embodiments further comprise retracting the catheter from the dysfunctional heart valve and expanding a balloon to expand the flutter valve. In specific embodiments, the flutter valve is expanded to a maximum diameter of 8-30 mm, including for example, 8 mm, 10 mm, 12 mm, 20 mm or 25 mm.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The terms “substantially” and “generally” are defined as largely but not necessarily wholly what is specified (and includes what is specified; e.g., substantially 90 degrees includes 90 degrees and generally parallel includes parallel), as understood by a person of ordinary skill in the art. In any disclosed embodiment, the terms “substantially,” “approximately,” and “about” may be substituted with “within [a percentage] of” what is specified, where the percentage includes .1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a vascular prosthetic assembly, or a component of such an assembly, that is configured in a certain way is configured in at least that way, but it can also be configured in other ways than those specifically described.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and other objects, features, and advantages of the invention will become apparent from the detailed description below and the accompanying drawings.

FIG. 1 is a perspective view of an expandable transcatheter valve according to an exemplary embodiment of the present disclosure.

FIG. 2 is a schematic view of the embodiment of FIG. 1 being inserted into a heart valve via a catheter.

FIG. 3 is a top view of a component used to construct the embodiment of FIG. 1.

FIG. 4 is a top view of the component of FIG. 3 modified during the construction of the embodiment of FIG. 1

FIG. 5 is a top view of the component of FIG. 3 modified during the construction of the embodiment of FIG. 1.

FIG. 6 is a perspective view of a component used to construct the embodiment of FIG. 1.

FIG. 7 is a graph showing data of pressure gradient versus time for increasing cycle counts during testing of an expandable transcatheter valve according to an exemplary embodiment of the present disclosure.

### DETAILED DESCRIPTION OF THE INVENTION

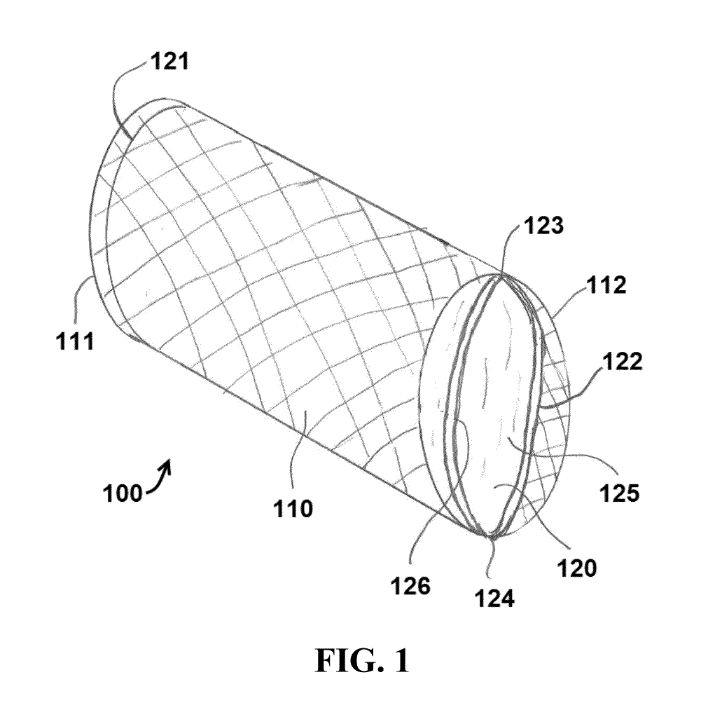

Referring initially to FIG. 1, an expandable transcatheter valve 100 comprises an expandable framework 110 and a tubular member 120 coupled to expandable framework 110. In particular embodiments, expandable framework 110 may be configured as a stent. In particular embodiments, expandable framework 110 is configured as a covered stent. In the embodiment shown, tubular member 120 comprises a first end 121 and tubular member 120 is coupled to expandable framework 110 proximal to first end 121. In addition, in this embodiment tubular member 120 comprises a second end 122. In the embodiment shown, tubular member 120 is coupled to expandable framework 110 at a first coupling location 123 and a second coupling location 124 proximal to second end 122. In the illustrated embodiment, first coupling location 123 is approximately 180 degrees radially from second coupling location 124 when tubular member is viewed looking toward second end 122.

In this embodiment, tubular member 120 is coupled to expandable framework 110 proximal to second end 122 at only first location 123 and second location 124 such that tubular member 120 comprises a first portion 125 and a second portion 126 between first and second locations 123 and 124. First portion 125 extends axially along tubular member 120 and extends clockwise radially from first coupling location 123 to second coupling location 124 when tubular member 120 is viewed looking toward second end 122. Second portion 126 also extends axially along tubular member 120, but extends clockwise radially from second coupling location 124 to first coupling location 123 when the tubular member 120 is viewed looking toward second end 122.

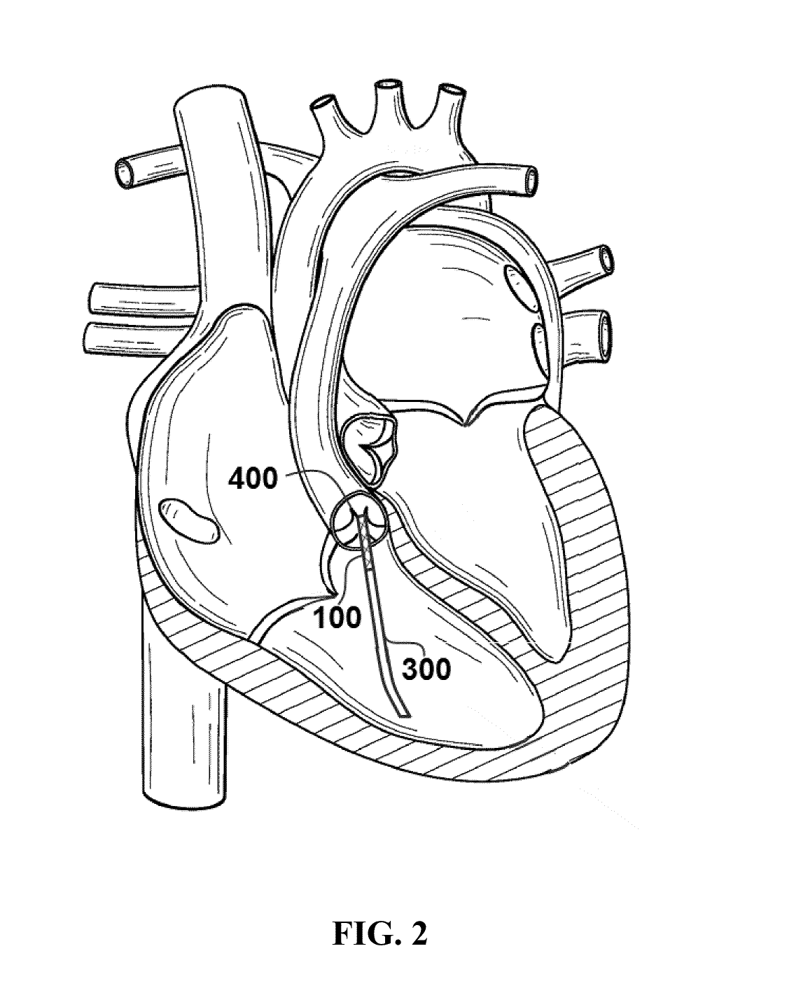

Referring now to FIG. 2, in exemplary embodiments, balloon expandable transcatheter valve 100 can be inserted via an angioplasty or balloon catheter 300 into a dysfunctional heart valve 400 of a patient and operate as a flutter valve (also known as a Heimlich valve). In particular embodiments, valve 100 can be placed by inserting an assembly comprising a catheter and valve 100 into dysfunctional heart valve 400 and retracting catheter 300 from dysfunctional heart valve 400. While valve 400 is shown as a mitral valve in FIG. 2, it is understood that in other embodiments, valve 400 may be a different valve (e.g. a mitral or tricuspid valve). For purposes of clarity, not all features of valve 100 are shown in FIG. 2. It is understood that the embodiment of valve 100 shown in FIG. 2 includes the features of those shown and described in other figures, including for example, FIG. 1.

The flutter valve configuration of valve 100 can be customized for insertion into dysfunctional heart valves for small children. In particular embodiments, valve 100 can be inserted into dysfunctional heart valves for smaller children, including those weighing more than 10 kg (including for example, patients weighing less than 40 kg, 30 kg, or 20 kg). In certain embodiments, valve 100 is configured such that it can be expanded and a second valve 100 can be placed.

During operation of valve 100, first portion 125 and second portion 126 of tubular member 120 are configured to allow fluid (e.g. blood) flow from first end 121 of tubular member 120 to second 122 end of tubular member 120 and restrict fluid flow from second end 122 of tubular member 120 to first end 121 of tubular member 120. Accordingly, valve 100 can be inserted into a dysfunctional heart valve that is not functioning properly in order to restore proper control of blood flow.

For example, when fluid flows from first end 121 to second end 122, first portion 125 and second portion 126 will be pushed away from each other (and toward expandable framework 110) by the fluid flow. In exemplary embodiments, tubular member 120 is secured to expandable framework proximal to first ends 111 and 121 at multiple locations around the circumference of tubular member 120. This can secure tubular member 120 to expandable framework 110 such that fluid will flow through tubular member 120 from first end 121 toward second end 122 without tubular member appreciably restricting flow.

However, if fluid pressure attempts to direct fluid flow from second end 122 toward first end 121, first and second portions 125 and 126 will be directed toward each other by the fluid flow and will restrict the fluid flow. The coupling of tubular member 120 to expandable framework 110 at only two locations proximal to second end 122 can allow first and second portions 125 and 126 to move inward to restrict flow.

While conventional heart valve replacement typically has utilized tricuspid replacement valves, the flutter (or Heimlich) valve configuration disclosed herein can provide certain advantages over a tricuspid arrangement. For example, the reduction in the number of moving parts in the valve can provide for an arrangement that is less expensive to manufacture. The cost of tricuspid valves can also be increased by factors such as reductions in the numbers of suitable animal (e.g. bovine) donors. One significant advantage is the ability to customize the valve to any size needed for the patient and therefore not be limited by the size of available biological scaffolding (e.g. existing devices such as those marketed as the Melody® valve).

In addition, the flutter valve configuration can provide fewer locations for fluid leakage and therefore reduced valve leakage. As previously mentioned, the flutter valve configuration can also allow the valve to be constructed in smaller sizes, suitable for use in smaller dysfunctional heart valves, including those of small children.

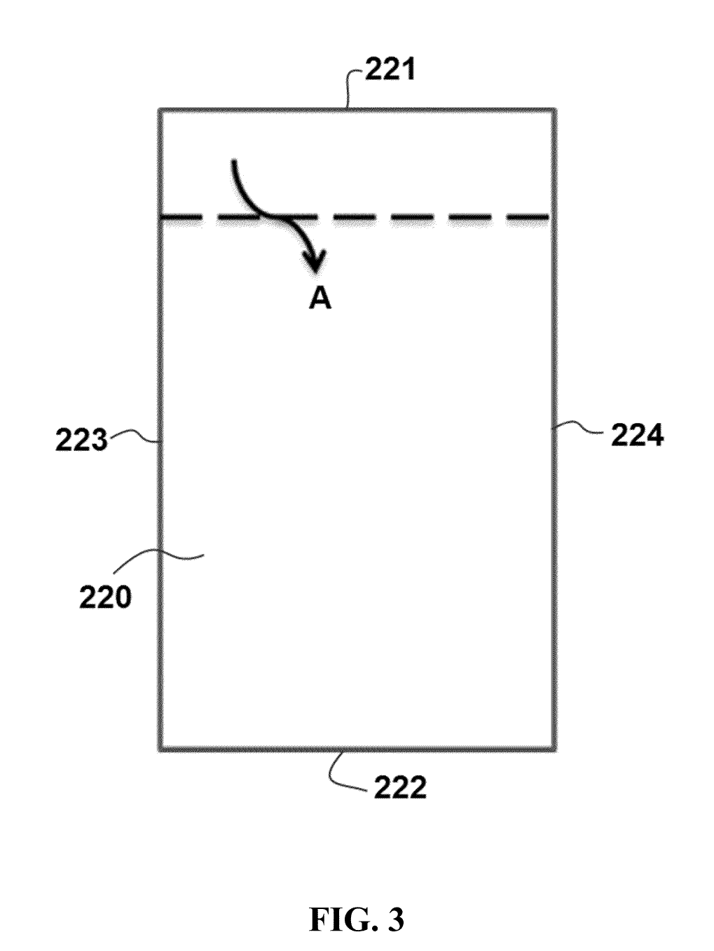

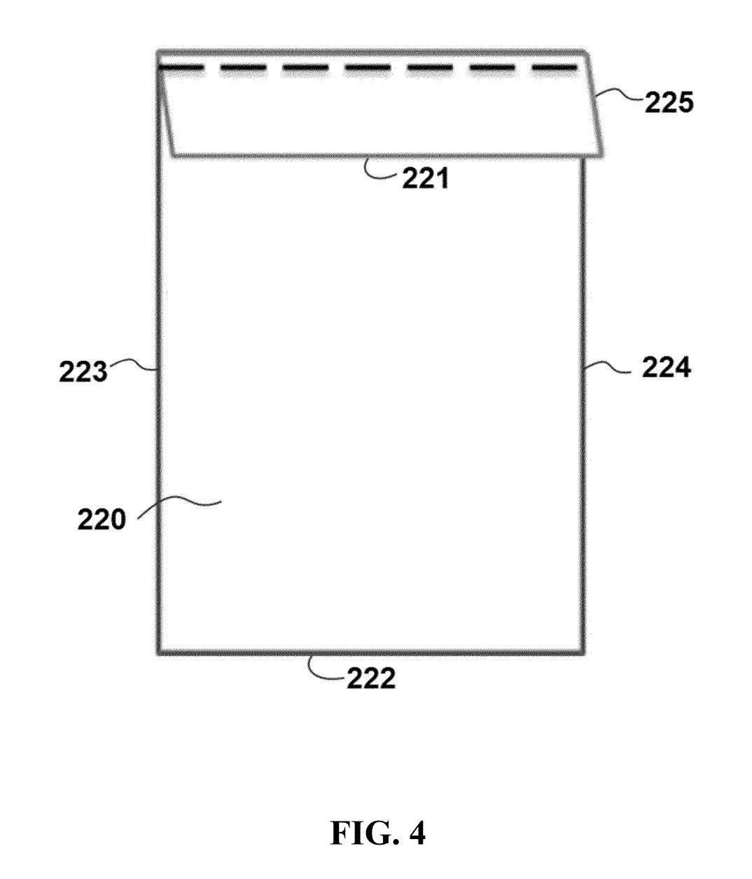

Referring now to FIGS. 3-6 one exemplary manner of constructing tubular member 120 is shown. It is understood that the method shown is merely one example, and that other methods of construction may be used. In this embodiment, tubular member 120 is constructed from a piece of rectangular material 220 that is flexible, suitable for suturing, biocompatible and able to withstand intercardiac pressures. In exemplary embodiments, material 220 may be formed from any suitable material, e.g. a biocompatible membrane. In the embodiment shown, rectangular material 220 comprises a first end 221, a second end 222, a first side 223 and a second side 224.

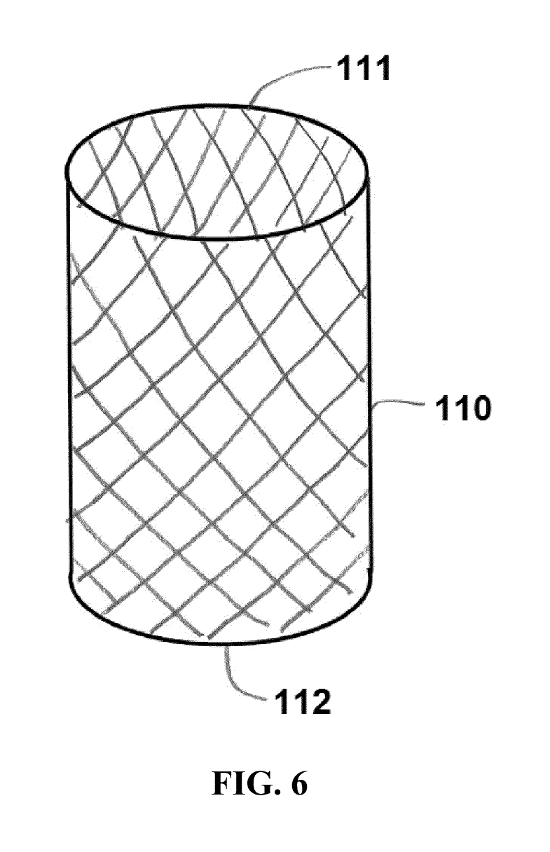

In specific embodiments, rectangular material 220 may be formed into a tubular (e.g. generally cylindrical) shape by coupling rectangular material 220 to an expandable framework 110 comprising a first end 111 and a second end 112 (shown in FIG. 6). Prior to coupling with material 220, expandable framework 110 can be dilated, for example, with an angioplasty catheter.

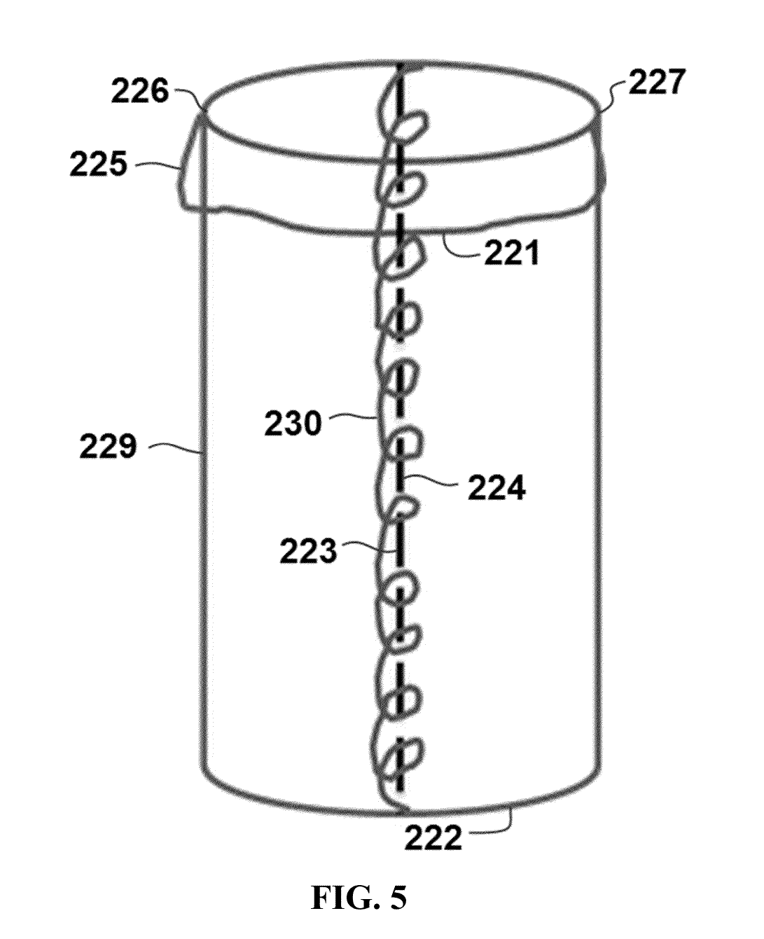

In one embodiment, rectangular material 220 may be formed into a generally cylindrical tubular shape 229 by folding first end 221 in the direction of arrow A to form a cuff 225 and suturing the two sides 223, 224 together via one or more sutures 230. In the embodiment shown in FIG. 5, a single suture 230 is shown. However, other embodiments may comprise multiple pieces of material 220 (e.g. two or more rectangular pieces) that can be coupled via multiple sutures to form generally cylindrical tubular shape 229. Material 220 can then be inserted into expandable framework 110 and cuff 225 sutured proximal to first end 111 of the expandable framework 110. As previously noted, in particular embodiments expandable framework 110 can be configured as a covered stent, which can reduce the likelihood of a paravalvular leak occurring between open cells of the stent and at the site of cuff 225. In the embodiment shown, cuff 223 can be sutured around the circumference of expandable framework 110 to secure material 220 to expandable framework 110. In this embodiment, second end 222 is coupled (e.g. sutured) to expandable framework at only two locations 226 and 227. In the embodiment shown, locations 226 and 227 are proximal to second end 112 of expandable framework when tubular shape 229 is coupled to expandable framework 110). In exemplary embodiments locations 226 and 227 are separated by approximately 180 degrees.

As described above, expandable framework 110 and the tubular member formed from material 220 can be inserted via a catheter into a dysfunctional heart valve. The assembly of expandable framework 110 and material 220 can act as flutter valve to allow fluid flow into first end 111 of expandable framework 110 (and cuff 225 of material 220) and to exit second end 112 of expandable framework 110 (and end 222 of material 220). The portions of material 220 located between locations 226 and 227 (e.g. each half of the circumference of tubular member 229) can act as a flutter valve to restrict fluid flow from second end 112 toward first end 111 of expandable framework 110.

In particular embodiments, material 220 may be a polytetrafluoroethylene (PTFE) material or other suitable material, including for example, materials available from CorMatrix®. Specific embodiments may be sized for particular applications. In certain embodiments, the diameter of expandable framework 110 can be expanded from a smaller diameter to a larger diameter (e.g. 8-30 mm in particular embodiments).

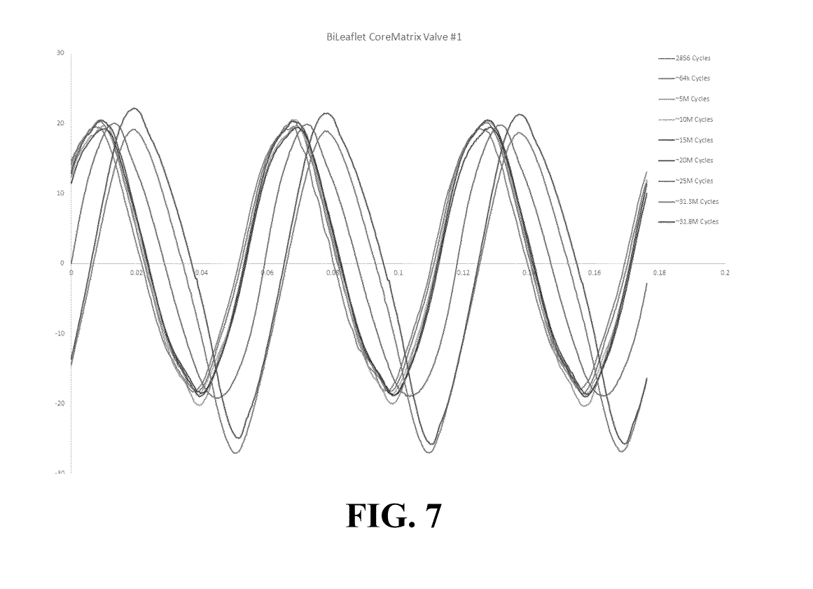

FIG. 7 is a graph is showing the pressure gradient versus time for increasing cycle counts of an exemplary embodiment of a valve during testing. In this test the valve was operated with the maximum pressure gradient of 20 mm Hg to achieve complete leaflet closure (amplitude of approximately 0.3 mm). In this test, the valve functioned for a total of approximately 30 million cycles. The mechanism of failure was that the valve leaflets were frayed along the valve edge.

In additional testing in a valve tester, another valve ran for a total of 65 million cycles. In that case, the mechanism of failure for was tearing of the suture at the distal end of the leaflet. In that case, it took a higher max pressure gradient to completely close the valve which also implicated a higher amplitude (approximately 0.7 mm).

Additional testing has also included a valve that was implanted in an animal. This valve did not have any insufficiency (leakage) and there was less than 10 mm pressure gradient across the valve which is acceptable from a hemodynamic standpoint. There were also no injuries to the adjacent structures during deployment.

It should be understood that the present devices and methods are not intended to be limited to the particular forms disclosed. Rather, they are to cover all modifications, equivalents, and alternatives falling within the scope of the claims.

The above specification and examples provide a complete description of the structure and use of an exemplary embodiment. Although certain embodiments have been described above with a certain degree of particularity, or with reference to one or more individual embodiments, those skilled in the art could make numerous alterations to the disclosed embodiments without departing from the scope of this invention. As such, the illustrative embodiment of the present devices is not intended to be limited to the particular forms disclosed. Rather, they include all modifications and alternatives falling within the scope of the claims, and embodiments other than the one shown may include some or all of the features of the depicted embodiment. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not to be interpreted as including means-plus- or step-plus-function limitations, unless such a limitation is explicitly recited in a given claim using the phrase(s) “means for” or “step for,” respectively.

### F. REFERENCES

The following references, to the extent that they provide exemplary procedural or other details supplementary to those set forth herein, are specifically incorporated herein by reference.

U.S. Pat. No. 6,458,153

J. Am. Coll. Cardiol. Intv. 2011;4: 721-32 ; Piazza, “Transcatheter Aortic Valve Implantation for Failing Surgical Aortic Bioprosthetic Valve.”

J. Am. Coll. Cardiol. 2005;46:360-5; Boudjemline, “Steps Toward the Percutaneous Replacement of Atrioventricular Valves.”

Annals of Biomedical Engineering, Vol. 40, No. 12, Dec. 2012; 2663-2673; Capelli, “Finite Element Strategies to Satisfy Clinical and Engineering Requirements in the Field of Percutaneous Valves.”

Ann Thorac Surg 2005;80:704-7; White, “A Stentless Trileaflet Valve From a Sheet of Decellularized Porcine Small Intestinal Submucosa.”

J. Thorac. Cardiovasc Surg 2009;137:1363-9; Zong, “Use of a novel valve stent for transcatheter pulmonary valve replacement: An animal study.”

J. Thorac. Cardiovasc Surg 2005;130:477-84; Ruiz, “Transcatheter placement of a low-profile biodegradable pulmonary valve made of small intestinal submucosa: A long-term study in a swine model.”

What is claimed is:

1. An expandable transcatheter valve comprising:
an expandable framework; and
a tubular member coupled to the expandable framework, wherein:
the tubular member comprises a first end and a second end;
the tubular member is coupled to the expandable framework proximal to the first end;
the tubular member is coupled to the expandable framework proximal to the second end at a first coupling location and at a second coupling location; and
the first coupling location is approximately 180 degrees radially from the second coupling location when the tubular member is viewed looking toward the second end.

2. The expandable transcatheter valve of claim 1 wherein the tubular member is coupled to the expandable framework proximal to the second end at only the first coupling location and the second coupling location.

3. The expandable transcatheter valve of claim 1 wherein:
the tubular member comprises a first portion and a second portion;
the first portion extends axially along the tubular member and extends clockwise radially from the first coupling location to the second coupling location when the tubular member is viewed looking toward the second end; and
the second portion extends axially along the tubular member and extends clockwise radially from the second coupling location to the first coupling location when the tubular member is viewed looking toward the second end.

4. The expandable transcatheter valve of claim 3 wherein the first portion and the second portion of the tubular member are configured to allow fluid flow from the first end of the tubular member to the second end of the tubular member and restrict fluid flow from the second end of the tubular member to the first end of the tubular member.

5. The expandable transcatheter valve of claim 4 wherein the first portion of the tubular member and the second portion of the tubular member form a flutter valve.

6. The expandable transcatheter valve of claim 1 wherein the expandable framework is configured as a wire stent.

7. The expandable transcatheter valve of claim 1 wherein the expandable framework is generally cylindrical.

8. The expandable transcatheter valve of claim 7 wherein the expandable framework is approximately 16-36 mm long.

9. The expandable transcatheter valve of claim 1 wherein:
the expandable framework is configured to expand from a smaller first diameter to a larger second diameter;
the expandable framework is biased towards the larger second diameter; and
the tubular member is initially coupled to the expandable framework when the expandable framework is expanded to the larger second diameter.

10. The expandable transcatheter valve of claim 8 wherein the second diameter is approximately 8-30 mm.

11. The expandable transcatheter valve of claim 1 wherein the tubular member is disposed within the expandable framework.

12. The expandable transcatheter valve of claim 1 further comprising an expandable catheter disposed within the expandable framework.

13. A method of inserting a flutter valve into a dysfunctional heart valve, the method comprising:
inserting an assembly comprising a catheter and a flutter valve into a dysfunctional heart valve; and
retracting the catheter from the dysfunctional heart valve, wherein the flutter valve is retained in the dysfunctional heart valve.

14. The method of claim 13 wherein the catheter has an outer diameter less than or equal to approximately 8 mm.

15. The method of claim 13 wherein the flutter valve comprises:
an expandable framework; and
a tubular member coupled to the expandable framework, wherein:
the tubular member comprises a first end and a second end;
the tubular member is coupled to the stent proximal to the first end;
the tubular member is coupled to the expandable framework proximal to the second end at a first coupling location and at a second coupling location; and
the first coupling location is approximately 180 degrees radially from the second coupling location when the expandable framework is viewed looking toward the second end.

16. The method of claim 13 wherein the dysfunctional heart valve is a pulmonary, tricuspid, or mitral valve.

17. The method of claim 13 wherein the dysfunctional heart valve is in a patient with a weight between 10 kg and 40 kg.

18. The method of claim 13 wherein the dysfunctional heart valve is in a patient younger than 10 years of age.

19. The method of claim 13 further comprising retracting the catheter from the dysfunctional heart valve and expanding a balloon to expand the flutter valve.

20. The method of claim 19 wherein the flutter valve is expanded to a maximum diameter of 8-30 mm.
